(19) United States
(12) Patent Application Publication
(10) Pub. No.: US 2011/0104073 A1
(43) Pub. Date: May 5, 2011

(54) Iron/Iron Oxide Nanoparticle and Use Thereof

(76) Inventors: Qi Zeng, Reading, IL (US); Ian Baker, Etna, NH (US)

(21) Appl. No.: 12/880,653

(22) Filed: Sep. 13, 2010

Related U.S. Application Data
(63) Continuation-in-part of application No. 12/522,938, filed on Aug. 26, 2009.
(60) Provisional application No. 60/885,512, filed on Jan. 18, 2007.

(51) Int. Cl. A61K 49/00; A61N 2/00; A61P 35/00; B05D 5/12; B82Y 5/00

(52) U.S. Cl. 424/9.32; 600/9; 428/403; 427/127; 977/773; 977/915; 977/927

(57) ABSTRACT

The present invention is a nanoparticle composition composed of an iron core with an iron oxide shell which is optionally coated with a micro-emulsion. The disclosed nanoparticle compositions are disclosed for use in hyperthermia treatment and imaging of cancer.

### INTRODUCTION

This application is a continuation-in-part of U.S. patent application Ser. No. 12/522,938, filed Aug. 26, 2009, which claims benefit from PCT/US2008/050557, filed Jan. 9, 2008 and U.S. Provisional Application Ser. No. 60/885,512, filed Jan. 18, 2007, the contents of which are incorporated herein by reference in their entireties.

This invention was made in the course of research sponsored by the National Institute of Standards and Technology (NIST Grant No. 60NANB2D0120). The government has certain rights in the invention.

### BACKGROUND OF THE INVENTION

Magnetic materials are known for use in producing hyperthermia in tumors. Fe₂O₃ nanoparticles, when injected into lymph nodes, have been shown to produce a temperature rise of 14° C. in an alternating magnetic field (Gilchrist, et al. (1957) Ann. Surgery 146:596-606). Polymer-coated superparamagnetic iron oxide (SPIO) nanoparticles have also been used to localize the hyperthermia to a tumor by tagging the nanoparticles with an antibody (Shinkai (2002) Biosci. Bioeng. 94:606).

In addition to the need for biocompatibility when used clinically, it is desirable to view the location of the nanoparticles in vivo prior to initiating treatment both to ensure productive therapy and to avoid normal tissue toxicity. Fine (<10 nm) SPIO nanoparticles serve the latter purpose since they can be observed by Magnetic Resonance Imaging (MRI) (Josephson, et al. (1988) Mag. Reson. Imag. 6:564-653). Nanoparticles with the highest specific absorption rate (SAR) value are of particular use. Having a large SAR value not only minimizes the dose of nanoparticles required for hyperthermia treatment, but is also a key parameter for the minimum size of tumor that can be treated. There also appears to be a limit to the concentration of nanoparticles that a cell can take up (Hergt, et al. (2004) J. Magn. Magn. Mater. 270:345-357).

The magnitude of the magnetic fields that have to be applied to SPIO nanoparticles to produce hyperthermia, at least in nude mice, can cause morbidity (Ivkov, et al. (2005) Clin. Cancer Res. 11(19 Suppl):7093s-7103s). It has been suggested (Andrä (1998) In: Magnetism in Medicine: A Handbook, Andrä and Nowak ed., Wiley-VCH, Berlin, p. 455) that for human use the product H·f should not be more than about 6×10⁶ Oe·Hz, where H is the applied field strength and f the frequency of the applied field. However, conventional nanoparticles can not meet these requirements.

### SUMMARY OF THE INVENTION

The present invention is a nanoparticle composition composed of an iron core and an iron oxide shell. In particular embodiments, the instant nanoparticle further includes a surfactant, or a silane layer and a biocompatible phospholipid coating.

The present invention is also a method for producing the nanoparticle composition of the present invention. The method involves reducing aqueous FeCl₃ within a NaBH₄ solution so that an iron core is formed and passivating the iron core to produce an iron oxide shell. In particular embodiments, the step of reducing aqueous FeCl₃ within a NaBH₄ solution further includes the use of a surfactant.

Methods for hyperthermia treatment of cancer and imaging cancer using the nanoparticles of the present invention are also provided.

### BRIEF DESCRIPTION OF THE DRAWINGS

FIG. 1 shows the X-ray diffraction patterns of nanocomposite particles produced using the indicated NaBH₄ flow rates with an NaBH₄ concentration of 0.2 M. Peaks corresponding to α-Fe and a possible Fe₃O₄ peak are indicated.

FIG. 2 shows the X-ray diffraction pattern for passivated nanocomposite particles with a NaBH₄ flow rate of 0.75 ml/minute.

FIG. 3 shows differential scanning calorimeter curves for three indicated NaBH₄ addition rates.

FIG. 4 shows X-ray diffraction patterns on powders obtained after total washing of CTAB. Panel A shows particles prepared in the presence of air and passivated. Panel B shows particles after they were annealed at 500° C. for 5 minutes under Ar. A-F3 peaks are shown.

FIG. 5 shows hysteresis loops for CTAB-coated Fe/Fe₃O₄ and Dextran-coated Fe₂O₃ dried powders at room temperature under a field of 8 kOe. The inset is a graph showing M-H loops for the same particles but under a field of 150 Oe, the same amplitude used for the heating test.

FIG. 6 shows temperature vs time for CTAB-coated Fe/Fe₃O₄ particles dispersed in methanol with a concentration of 5 mg/ml under an alternating magnetic field of 150 Oe and 250 kHz. Data for Dextran-coated Fe oxide particles with the same concentration, but dispersed in water, are given for comparison. The drop of temperature was due to magnetic field being turned off.

FIG. 7 shows R2* decay constant vs particle concentration for iron oxide (FIG. 7A) and Fe/Fe oxide (FIG. 7B) nanoparticles.

FIG. 8 shows a schematic of surface engineering procedures for the Fe/Fe₃O₄ nanoparticles. Surface engineering includes HMDS silanization (FIG. 8A) and PC assembly onto the Fe/Fe₃O₄ nanoparticle with a hydrophobic surface to form biocompatible coating (FIG. 8B).

FIG. 9 shows a plot of temperature versus time for Fe/Fe₃O₄/HMDS/PC nanoparticles and Dextran-coated Fe oxide particles dispersed in water at a concentration of 6 mg/ml under an alternating magnetic field of 150 Oe at 250 kHz.

### DETAILED DESCRIPTION OF THE INVENTION

The present invention relates to magnetic nanoparticles and the use of the same in the treatment cancer. A nanoparticle of the present invention is composed of a metallic core and a metal oxide shell. The instant nanoparticles are an improvement over conventional magnetic nanoparticles magnetic as the metallic core provides for heating in hyperthermia applications and the metal oxide shell provides MRI contrast for determining the localization of the nanoparticle.

Given the improved characteristics of the disclosed nanoparticles, the present invention specifically embraces a Fe/Fe₃O₄ core/shell nanoparticle synthesized by reduction of aqueous FeCl₃ within a NaBH₄ solution with or without micro-emulsions. Advantageously, Fe/Fe₃O₄ core/shell nanoparticles of the present invention have large SAR values thereby minimizing the dose of nanoparticles required for hyperthermia treatment. Moreover, in the presence of micro-emulsions, smaller, single domain particles (10-15 nm) with a narrow size distribution are obtained with a maximum SAR of 345 W/g at an alternating field of 150 Oe and 250 kHz.

While iron and iron oxide were employed in the production of the exemplary nanoparticles of this invention, it is contemplated that the core of the instant nanoparticle can be composed of one metal or can be formed of more than one type of atom. For example, the nanoparticle core can be a composite or an alloy. Exemplary metals of use include Au, Ag, Pt, Cu, Gd, Zn, Fe and Co. As such, nanoparticle cores can be formed from alloys including Au/Fe, Au/Cu, Au/Gd, Au/Zn, Au/Fe/Cu, Au/Fe/Gd, Au/Fe/Cu/Gd and the like.

Likewise, while iron oxide was used to produce the shell of the instant nanoparticle, other magnetic metal oxides can be employed. Other oxides include those of cobalt or nickel; oxides of intermetallic compounds (e.g., CoPt, FePt, etc.); and oxides of alloys of such metals (e.g., Co/Ni, Co/Fe, Ni/Fe, Co/Fe/Ni, etc.).

Nanoparticles of the present invention can be synthesized as disclosed herein by reducing aqueous FeCl₃ within a NaBH₄ solution so that an iron core is formed and passivating the iron core to produce an iron oxide shell. An exemplary method for passivation is exposure of the iron core to Ar+air atmosphere. In certain embodiments, the step of reducing aqueous FeCl₃ within a NaBH₄ solution further includes a surfactant. For the purposes of the present invention, a surfactant is an organic compound that lowers the surface tension of a liquid. In particular embodiments, a surfactant of this invention is not a phospholipid. Surfactants include, but are not limited to, amines, amine oxides, ethers, quaternary ammonium salts, betaines, sulfobetaines, polyethers, polyglycols, polyethers, organic esters, alcohols, phosphines, phosphates, carboxylic acids, carboxylates, thiols, sulfonic acids, sulfonates, sulfates, ketones, silicones and combinations thereof. More specific examples of surfactants include, but are not limited to, methyl laureate, methyl oleate, dimethyl succinate, propylenglycol, hexadecylamine, ethyl dimethyl amine oxide, cetyl trimethyl ammonium bromide, poly n-vinyl pyrrolidone, n-butanol, tributyl phosphine, tributyl phosphate, trioctyl phosphine oxide, hexadecyl thiol, dodecyclbenzene sulfonate, diisobutyl ketone and dodecylhexacyclomethicone and combinations thereof. In one embodiment, the surfactant is CTAB. In another embodiment, the surfactant is CTAB, with n-butanol as co-surfactant. In certain embodiments, the surfactant and co-surfactant are combined with an oil phase (e.g., n-octanol) to form a micro-emulsion.

The mean diameter of the present nanoparticle is generally between 0.5 and 100 nm, more desirably between 1 and 50 nm, and most desirably between 1 and 20 nm. The mean diameter can be measured using techniques well-known in the art such as transmission electron microscopy (TEM).

Some embodiments of the present invention embrace nanoparticles which are linked or conjugated to one or more antibodies. Such antibodies can be specific for any tumor antigen and may also have a therapeutic effect. Desirably, the antibodies are attached covalently to the nanoparticles. Protocols for carrying out covalent attachment of antibodies are routinely performed by the skilled artisan. For example, conjugation can be carried out by reacting thiol derivatized antibodies with the nanoparticle under reducing conditions. Alternatively, the antibodies are derivatized with a linker, e.g., a disulphide linker, wherein the linker can further include a chain of ethylene groups, a peptide or amino acid groups, polynucleotide or nucleotide groups.

Antibodies of use in accordance with the present invention include an antibody (e.g., monoclonal or polyclonal) or antibody fragment which binds to a protein or receptor which is specific to a tumor cell. Preferably, the antibody fragment retains at least a significant portion of the full-length antibody's specific binding ability. Examples of antibody fragments include, but are not limited to, Fab, Fab′, F(ab′)₂, scFv, Fv, dsFv diabody, or Fd fragments. Exemplary tumor-specific antibodies for use in the present invention include an anti-HER-2 antibody (Yamanaka, et al. (1993) Hum. Pathol. 24:1127-34; Stancovski, et al. (1994) Cancer Treat Res. 71:161-191) for targeting breast cancer cells, an anti-A33 antigen antibody for targeting colon or gastric cancer (U.S. Pat. No. 5,958,412), anti-human carcinoembryonic antigen (CEA) antibody for targeting carcinomas (Verstijnen, et al. (1986) Anti-Cancer Research 6:97-104), HMFG2 or H17E2 antibodies for targeting breast cancer (Malamitsi, et al. (1988) J. Nucl. Med. 29:1910-1915), and bispecific monoclonal antibodies composed of an anti-histamine-succinyl-glycine Fab′ covalently coupled with an Fab′ of either an anticarcinoembryonic antigen or an anticolon-specific antigen-p antibody (Sharkey, et al. (2003) Cancer Res. 63(2):354-63).

In some embodiments, the nanoparticles can further include a radionuclide for therapeutic applications (i.e., interstitial therapy). Examples of radionuclides commonly used in the art that could be readily adapted for use in the present invention include ^{99m}Tc, which exists in a variety of oxidation states although the most stable is TcO⁴⁻; ³²P or ³³P; ⁵⁷Co; ⁵⁹Fe; ⁶⁷Cu which is often used as Cu²⁺ salts; ⁶⁷Ga which is commonly used as a Ga³⁺ salt, e.g., gallium citrate; ⁶⁸Ge; ⁸²Sr; ⁹⁹Mo; ¹⁰³Pd; ¹¹¹In, which is generally used as In³⁺ salts; ¹²⁵I or ¹³¹I which is generally used as sodium iodide; ¹³⁷Cs; ¹⁵³Gd; ¹⁵³Sm; ¹⁵⁸Au; ¹⁸⁶Re; ²⁰¹Tl generally used as a Tl⁺ salt such as thallium chloride; ³⁹Y³⁺; ⁷¹Lu³⁺; and ²⁴cr²⁺. The general use of radionuclides in radiation therapy is well-known in the art and could readily be adapted by the skilled person for use in the aspects of the present invention. The radionuclides can be employed most easily by doping the nanoparticles or including them as labels present as part of the antibody immobilized on the nanoparticles.

In other embodiments, the nanoparticles can be linked to a therapeutically active substance such as a tumor-killing drug or, as indicated above, a radionuclide for providing interstitial radiation at the site of the tumor. The magnetic properties of the nanoparticles can also be used to target tumors, by using a magnetic field to guide the nanoparticles to the tumor cells.

In still further embodiments, the surface of the instant nanoparticles is modified. As one example of a surface modification, the surface of the instant nanoparticle is coated with a silane layer. Silanization of the nanoparticle is achieved by removing the surfactant from the nanoparticle and contacting the nanoparticle with a silane coupling agent such as an organochloro- or organoalkoxy-silane. As described herein, a surfactant can be removed by strong washing, sintering, replacement by other reaction agents, or, using a mixture of TMAH and isopropanol, as exemplified herein. Silanization can occur within various environmental conditions, and through exposure to both liquid and vapor phases of the silane coupling agent. Silane coupling agents that may be used include, but are not limited to, disilazane, trichlorosilane, trimethoxy silane, triethoxy silane, silanol, siloxane, disiloxane, n-dodecyltrichlorosilane, and octyltrichlorosilane. In a particular embodiment, the surface coating is hydrophobic. In accordance with this embodiment, the silanizing agent is, e.g., trimethyl chlorosilane, hexamethyldisilazane (“HMDS” or (CH₃)₃SiNHSi(CH₃)₃) or another dimethylalkylchlorosilane in which the alkyl substituent may contain from one to about eighteen carbon atoms, preferably about one to four carbon atoms.

As an additional surface modification, the instant nanoparticle can be coated with a biocompatible phospholipid, e.g., as exemplified herein. The phospholipid is biocompatible in the sense that it is tolerated in vivo without toxic effects. Phospholipids are defined as amphiphile lipids which contain phosphorus. A phospholipid of this invention can be a zwitterionic phospholipid, a saturated phospholipid, a hydrogenated phospholipid, a pure phospholipid; or a mixture of such phospholipids. Phospholipids play an important role in nature, in particular, as double layer-forming constituents of biological membranes. Phospholipids which are chemically derived from phosphatidic acid occur widely and are also commonly used for pharmaceutical purposes. This acid is a usually (doubly) acylated glycerol-3-phosphate in which the fatty acid residues may be of different length. The derivatives of phosphatidic acid include, for example, the phosphocholines or phosphatidylcholines, in which the phosphate group is additionally esterified with choline, furthermore phosphatidyl ethanolamines, phosphatidyl inositols etc. Lecithins are natural mixtures of various phospholipids which usually have a high proportion of phosphatidyl cholines. Suitable phospholipids also include phospholipid mixtures, which are extracted in the form of lecithin from natural sources such as soya beans or chickens egg yoke. Purified, enriched or partially synthetically prepared medium- to long-chain zwitterionic phospholipids are also embraced by this invention. Examples for enriched or pure compounds are dimyristoyl phosphatidyl choline (PC), dimyristoyl phosphatidyl choline (DMPC), distearoyl phosphatidyl choline (DSPC) and dipalmitoyl phosphatidyl choline (DPPC). Other examples of phospholipids include phosphoryl ethanolamine, phosphatidyl ethanolamine, phosphoethanolamine, and phosphatidyl serine. Alternatively, phospholipids with oleyl residues and phosphatidyl glycerol without choline residue are suitable for some embodiments and applications of the invention.

The following examples of application for the instant nanoparticles are provided by way of illustration and should not be construed to limit the wide applicability of the technologies described herein.

The magnetic properties of the nanoparticles of the invention can be exploited in cell separation techniques thereby eliminating the need for columns or centrifugation. By adding the nanoparticles to a cell suspension and separating the particle-bound cells from the rest of the suspension by application of a magnetic field, a highly pure population of tumor cells can be obtained quickly and easily. This is a highly sensitive as well as efficient method which can be used in many applications, for example in diagnosis of tumors by testing body fluids for the presence of tumor cells.

Advantageously, the instant nanoparticles can be used to treat cancer. Magnetic nanoparticles can be used in the hyperthermic treatment or combined hyperthermic and radiation treatment of tumors, in which magnetic nanoparticles are injected into tumors and subjected to a high frequency AC or DC magnetic field. Alternatively, near infrared light can be used. The heat thus generated by the relaxation magnetic energy of the magnetic material kills the tumor tissue around the particles. In vitro experiments with magnetic fluids have confirmed their excellent power absorption capabilities, attributable to the large number and surface of heating elements (Jordan, et al. (1993) Int. J. Hyperthermia 9(1):51-68). Advantageously, the instant nanoparticles can be localized by MRI given the magnetic properties of the iron oxide shell. To demonstrate efficacy of the instant nanoparticles, cell death or long-term toxicity is determined with cultured cells exposed to the instant magnetic nanoparticles alone or in an alternating magnetic field. Cytotoxicities of the cultured cells are also detected after magnetic hyperthermia treatments.

Moreover, by coating the instant nanoparticles with a surfactant, said nanoparticles can be taken up intracellularly by differential endocytosis (Jordan, et al. (1996) Int. J. Hyperthermia 12(6):705-722; Jordan, et al. (1999) J. Magn. Magn. Mater. 194:185-196), thereby providing intracellular hyperthermia.

Radiation treatment can delivered by a radiation source such as an external X-ray applicator (e.g., Gulmay Medical D3-225) (see, e.g., Johannsen, et al. (2006) Prostate 66:97-104), via a temporary radiation source placed temporarily in the tumor, alternatively by a radionuclide associated with the nanoparticle as disclosed herein.

By conjugating the nanoparticles with an antibody or antibodies that specifically bind to tumor antigens, tumor cells can be specifically targeted using the instant nanoparticles thereby improving the therapeutic ratio. This also allows tumors not easily reached by injection to be targeted by the therapeutic particles, and avoids killing of normal healthy cells. Moreover, the antibody-conjugated particles of the present invention can be delivered specifically to tumor cells so even tumor cells which have moved away from the original tumor site can be targeted for therapy.

The nanoparticles described herein can be formulated in pharmaceutical compositions, and administered to patients in a variety of forms. Thus, the nanoparticles can be used as a medicament for tumor targeting and hyperthermic/radiation therapies, or for in vivo cell and tissue labeling.

Pharmaceutical compositions for oral administration can be in tablet, capsule, powder or liquid form. A tablet can include a solid carrier such as gelatin or an adjuvant or an inert diluent. Liquid pharmaceutical compositions generally include a liquid carrier such as water, petroleum, animal or vegetable oils, mineral oil or synthetic oil. Physiological saline solution, or glycols such as ethylene glycol, propylene glycol or polyethylene glycol can be included. Such compositions and preparations generally contain at least 0.1 wt % of the compound.

Parenteral administration includes administration by intravenous, cutaneous or subcutaneous, nasal, intramuscular, intraocular, transepithelial, intraperitoneal and topical (including dermal, ocular, rectal, nasal, inhalation and aerosol), and rectal systemic routes. For intravenous, cutaneous or subcutaneous injection, or injection at the site of affliction (i.e., intratumoral), the active ingredient will be in the form of a parenterally acceptable aqueous solution which is pyrogen-free and has suitable pH, isotonicity and stability. Those of relevant skill in the art are well able to prepare suitable solutions using, for example, solutions of the compounds or a derivative thereof, e.g., in physiological saline, a dispersion prepared with glycerol, liquid polyethylene glycol or oils.

In addition the pharmaceutical compositions can include one or more of a pharmaceutically acceptable excipient, carrier, buffer, stabilizer, preservative or anti-oxidant or other materials well-known to those skilled in the art. Such materials should be non-toxic and should not interfere with the efficacy of the active ingredient. The precise nature of the carrier or other material may depend on the route of administration, e.g., orally or parenterally.

Liquid pharmaceutical compositions are typically formulated to have a pH between about 3.0 and 9.0, wherein the pH of a composition can be maintained by the use of a buffer such as acetate, citrate, phosphate, succinate, Tris or histidine, typically employed in the range from about 1 mM to 50 mM. The pH of compositions can otherwise be adjusted by using physiologically acceptable acids or bases.

Preservatives are generally included in pharmaceutical compositions to retard microbial growth, extending the shelf-life of the compositions and allowing multiple use packaging. Examples of preservatives include phenol, meta-cresol, benzyl alcohol, para-hydroxybenzoic acid and its esters, methyl paraben, propyl paraben, benzalconium chloride and benzethonium chloride. Preservatives are typically employed in the range of about 0.1 to 1.0% (w/v).

Desirably, the pharmaceutically compositions are given to an individual in a “prophylactically effective amount” or a “therapeutically effective amount” (as the case may be, although prophylaxis may be considered therapy), this being sufficient to show benefit to the individual. Typically, this will be to cause a therapeutically useful activity providing benefit to the individual. The actual amount of the compounds administered, and rate and time-course of administration, will depend on the nature and severity of the condition being treated. Prescription of treatment, e.g., decisions on dosage, etc., is within the responsibility of general practitioners and other medical doctors, and typically takes account of the cancer to be treated, the condition of the individual patient, the site of delivery, the method of administration and other factors known to practitioners. Examples of the techniques and protocols mentioned above can be found in Remington: The Science and Practice of Pharmacy, Alfonso R. Gennaro, editor, 20th ed. Lippincott Williams & Wilkins: Philadelphia, Pa., 2000.

The invention is described in greater detail by the following non-limiting examples.

### Example 1

### Materials and Methods

Fe₂O₃ nanoparticles were purchased from Alfa Aesar. Fe/Fe oxide nanoparticles were synthesized by reduction of aqueous solutions of FeCl₃ within a NaBH₄ solution, with or without the presence of a micro-emulsion. For synthesis of Fe/Fe oxide nanoparticle without a micro-emulsion, a typical procedure (carried out in an inert atmosphere or in aerobic conditions, at room temperature and ambient pressure) was started with dropwise addition of NaBH₄ into a vigorously stirred FeCl₃ solution. At the beginning of the reaction, the solution turned to a blackish color due to the precipitation of particles. The precipitates were washed with de-ionized (DI) water and acetone. Prior to use, DI water and acetone were purged with Ar for several hours to get rid of the oxygen. Anhydrous FeCl₃ purchased from Alpha Aesar was stored in glove box until used. Aqueous solutions of FeCl₃ were prepared immediately before nanoparticle synthesis using prepurged DI water.

After washing, the specimens were subjected to a few hours in an Ar+air atmosphere to passivate the surface. Since the particles were strongly pyrophoric, care was taken to spread the particles gently. Passivation or further annealing at low temperature (150-300° C.) produced a Fe/Fe₃O₄ core/shell structure. Some powder samples were heated in a gas flow of Ar between 400° C.-600° C. to make the particles grow and/or crystallize.

Coated Fe/Fe oxide nanoparticles were prepared using water-in-oil micro-emulsion with cetyl trimethyl ammonium bromide (CTAB) as the surfactant, n-butanol as the co-surfactant, n-octane as the oil phase Pillai and Shah (1996) J. Magn. Magn. Mater. 163:243), and an aqueous FeCl₃ or NaBH₄ solution as the water phase. Micro-emulsions were prepared by dissolving the two salt solutions into a CTAB/n-butanol/n-octane solution. Two micro-emulsions (I and II) with identical compositions (see Table 1) but different aqueous phases were used. These two micro-emulsions were then mixed under constant stirring. Due to the frequent collisions of aqueous cores of water-in-oil micro-emulsions, the reacting species in the two micro-emulsions came in contact with each other, leading to the precipitation of Fe within the aqueous micro-droplets of the micro-emulsion (Eicke, et al. (1976) Colloid Interface Sci. 56:168). Since the two micro-emulsions were of identical compositions, differing only in the nature of the aqueous phases, the micro-emulsion did not destabilize upon mixing. As the surfactant monolayer provided a barrier restricting the growth of the particles, it also hindered coagulation of the particles and therefore monodisperse particles are obtainable.

TABLE 1

| | Micro- | Micro- | Weight |
|---|---|---|---|
| | emulsion I | emulsion II | percentage (%) |
| Aqueous Phase | 0.08 M FeCl₃ | 0.2 M NaBH₄ | 34 |
| Surfactant | CTAB | CTAB | 12 |
| Co-Surfactant | n-butanol | n-butanol | 1 |
| Oil Phase | n-octane | n-octane | 44 |

The precipitated particles were separated using high speed centrifugation. The precipitate was then washed in methanol to remove any oil and surfactant from the particles. The particles were then re-dispersed in methanol. The concentration of dispersed solution was determined from the measured Ms of the solution sample using the Ms of uncoated dry powders. Powder samples were obtained by coagulating the colloids with acetone then washing with distilled water and acetone several times to totally remove the CTAB. The precipitates were then dried in flowing Ar at 100° C.

Phase analysis and the crystallite size were determined via a Siemens D5000 diffractometer using Cu-Kα radiation. The particle size and shape as well as the core-shell structure were determined by an FEI F20 field emission gun transmission electron microscopy (TEM). Thermal analysis was performed using a Perkin Elmer DSC 7 differential scanning calorimeter. The quasi-static magnetic properties of the nanoparticles were measured using a Lakeshore model 7300 vibrating sample magnetometer (VSM).

The SARs of the particles were analyzed by placing either 0.4 ml of solution or solid sample in a well-insulated, nonmetallic container, which was then placed in an air-cooled, 11 mm diameter×35 mm long magnetic excitation coil. For solid samples, the nanoparticles were dispersed uniformly in Epofix® resin and the resulting mixture solidified at room temperature. The dispersion generally resulted in a particle/resin ratio of less than 4% in weight, making the dipole-dipole interparticle interaction negligible. Although the dimension of the specimens was much shorter than the homogeneous magnetic zone along the z-axis of the coil, care was taken to maintain the suspension in a constant field zone within the coil. Heating tests were performed using a Hafler P7000 power amplifier to drive a resonant network composed of the magnetic coil and polypropylene capacitors, which were used to achieve a real input impedance matched to the amplifier capability for maximum efficiency. A Tektronix 60 MHz AC current probe was used with an Agilent Infinium digital oscilloscope to measure the current. The field strength was determined from the peak current. An alternating magnetic peak field strength of 150 Oe and a frequency of 250 kHz were applied. These field parameters were chosen to satisfy the criteria established in the art for use on the human body (Baker, et al. (2006) J. Appl. Phys. 99 (8):08H106).

The increase in solution temperature was recorded as a function of time by a fiber temperature sensor (Luxtron Corporation, Santa Clara, Calif.). As a control, the temperature rise of the same amount of DI water and pure resin without nanoparticles present was also measured and subtracted from the temperature rise measured for the nanoparticles. SAR (W/g) per unit mass of ferromagnetic material was defined by:

$$SAR = ( c \cdot {m}_{t} \cdot {( \frac{Δ T}{Δ t} )}_{max} ) / {m}_{p}$$

where c is the specific heat capacity of the specimen, mₚ is the mass of the particles, mₜ is the total mass of the specimen. T is temperature, and t is time. This means the data were normalized with respect to the particle mass. For the particle/resin composite, the heat capacity of the system was calculated as follows:

c=(Wₚcₚ+Wᵣₑₛᵢₙcᵣₑₛᵢₙ)/(Wₚ+Wᵣₑₛᵢₙ)

where Wₚ is the mass of Fe oxides or Fe. The following values of c were used: cᵣₑₛᵢₙ=1.4 J/(k.g); c_{Fe2O3}=0.75 J/(k.g); c_{Fe}=0.44 J/(k.g); cₘₑₜₕₐₙₒₗ 2.55 J/(k.g) (Specific heat capacity, From Wikipedia, the free encyclopedia).

### Example 2

### Characterization of Nanoparticles

To achieve the development of sufficient heat at the lowest possible frequency and the smallest external magnetic field strength, iron/iron oxide nanoparticles were produced. The iron/iron oxide combination was selected because iron has a high M_{S} (>210 emu/g), while the M_{S} of iron oxides are 90 emu/gram. Theoretically, the hysteresis power loss to heat is given by the frequency times the integral of B·dH over a closed loop, where B is the inductive magnetization. As such, Fe nanoparticles can have high enough coercivities for hyperthermia with limited applied field amplitudes, and since B for iron is more than twice that of iron oxides, the power losses of a single domain Fe particle can be more than twice that of an iron oxide particle.

While ferromagnetic particles such as Fe can be imaged with MRI, the contrast is much less than the contrast that can be achieved with SPIO nanoparticles. Accordingly, the instant nanoparticles combine a single-domain core of pure iron covered with 3-4 nm of iron oxide. In this regard, the instant nanoparticle achieves a higher SAR of pure iron (compared to iron oxides) for heating, while using the film of superparamagnetic iron oxide for imaging of the nanoparticles.

The instant Fe/Fe oxide nanoparticles, were produced by reduction of an aqueous solution of FeCl₃ within a NaBH₄ solution, or, using a water-in-oil micro-emulsion with CTAB as the surfactant. The reduction was performed either in an inert atmosphere or in air, and passivation with air was performed to produce the Fe/Fe₃O₄ core/shell composite. Particles with different sizes and magnetic properties were produced by varying the flow rate of the NaBH₄ addition into a FeCl₃ solution (0.75 ml/minute, 5 ml/minute and 50 ml/minute) while keeping the concentration of FeCl₃ and NaBH₄ solutions constant at 0.08 M and 0.2 M, respectively. Transmission electron microscopy (TEM) electron micrographs indicated that the particles had a nearly spherical shape with a mean size of ˜40 nm when the flow rate was 50 ml/minute. The tendency of the particles to form a long chain-like structure was also observed. The chain could be due to dipolar coupling, favoring a head-to-tail orientation (Chantrell, et al. (1980) J. Phys. D: Appl. Phys. 13:1119). It was found that decreasing the NaBH₄ flow rate increased the particle size substantially. A very slow flow rate (0.75 ml/minute) was found to decrease the H_{C}, but it also increased the particle size to more than 200 nm. The latter samples also demonstrated a particle-aggregate morphology. Thus, decreasing the addition rate of NaBH₄ was not a suitable way to vary magnetic properties to obtain a better heating effect.

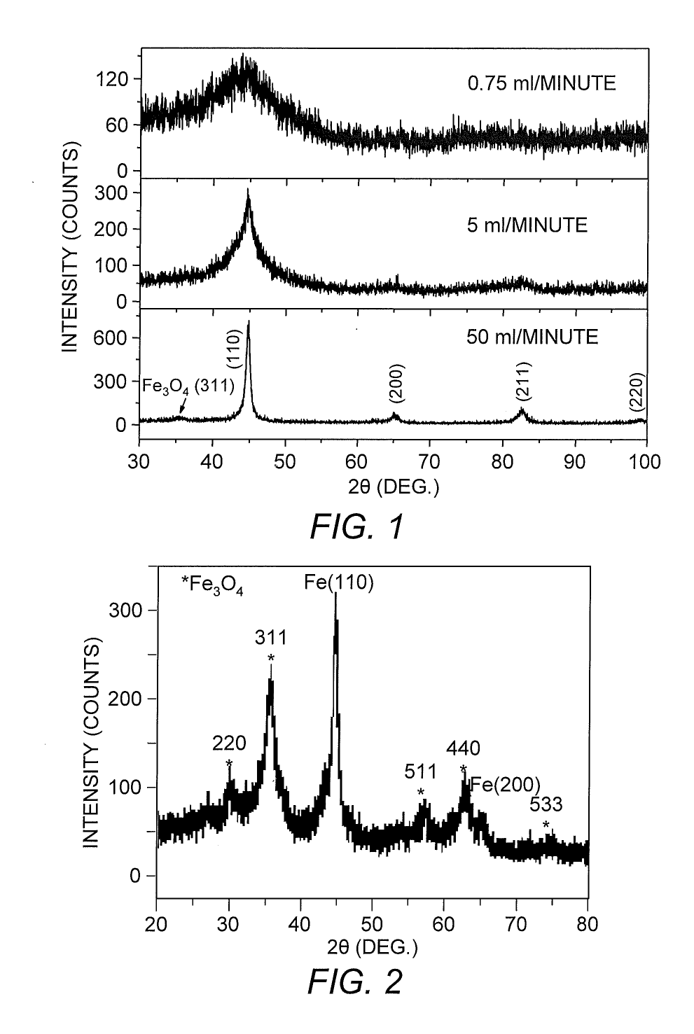

The X-ray diffraction patterns of the nanoparticles produced with the three different NaBH₄ flow rates are shown in FIG. 1. The slowest flow rate sample showed a typical amorphous or extremely fine nanocrystalline structure. With increasing flow rate the peaks became sharper. When the NaBH₄ flow rate was 50 ml/minute, the sample showed a pure nanocrystalline phase with grain size of ˜25 nm (determined by Scherrer formula from X-ray line broadening) and the peaks could be clearly identified as b.c.c. α-Fe. This grain size measured by X-ray diffraction was smaller than the particle size determined by TEM (40 nm), which indicated that the particles were polycrystalline. None of Fe oxide peaks were definitely detected by X-ray diffraction possibly because they were too broad and had low intensities. However, when the reduction reaction was performed in air, and the passivation was undertaken for a very long time, X-ray diffraction analysis indicated that these particles were composed of Fe and Fe₃O₄ (see FIG. 2).

Thermal analysis was also conducted using a differential scanning calorimeter (DSC). DSC curves for the nanoparticles are shown in FIG. 3. The slow and medium flow rate materials showed a sharp exothermic peak at 471° C. and 497° C. upon heating, respectively. The high flow rate (50 ml/minute) sample showed more complicated behavior with several phase transformations. In addition, the transition temperatures tended to increase and transition energy tended to decrease with increasing flow rate.

To vary the particle size and, hence, the magnetic properties, the concentration of NaBH₄ was varied while the concentration of FeCl₃ was held constant (0.8 M), or through subsequent heat treatment. Although the crystalline grain size decreased when decreasing the NaBH₄ concentration from 0.5 M to 0.025 M, the particle size did not significantly change (40-50 nm), see Table 2. However, the particle size distribution increased. Some particles had a size of more than 100 nm. Heat treatment varied dramatically (Table 2), however, the particle size was maintained at nanoscale. It is possible that the Fe₃O₄ coating prevented form coarsening.

TABLE 2

| | | | Particle | | SAR (W/g) |
|---|---|---|---|---|---|
| | MS_{(8 kOe)} | | Size | | (150 Oe/250 |
| Condition* | (emu/g) | H_{C}(Oe) | (nm) | Wt % | kHz) |
| 0.025 M | 133 | 288 | 40-50 | 10.02 | 1.9 |
| 0.05 M | 146 | 329 | 40-50 | 7.14 | 3.3 |
| 0.1 M | 148 | 451 | 40-50 | 2.58 | 5.1 |
| 0.2 M | 157 | 66 | 200 | 3.38 | 11.6 |
| (0.75 mL/min) | | | | | |
| 0.2 M | 169 | 580 | 40 | 4.05 | 4.5 |
| (5 mL/min) | | | | | |
| 0.2 M | 137 | 581 | 40 | 2.98 | 3.9 |
| (50 mL/min) | | | | | |
| 0.5 M | 133 | 617 | 40 | 2.58 | 6.4 |
| 0.2 M | 168 | 74 | 80 | 1.0 | 31.3 |
| (5 mL/min) | | | | | |
| 600° C./5 min | | | | | |
| 0.2 M | 194 | 462 | 50 | 3.72 | 5.7 |
| (5 mL/min) | | | | | |
| 500° C./5 min | | | | | |
| Fe₂O₃ (20 nm) | 57 | 101 | 25 | 1.84 | 8.8 |
| Fe₂O₃ (9 nm) | 51 | 3.5 | 9 | 3.33 | 6.9 |

* The molar concentration is for NaBH₄, the flow rate is indicated in parentheses.

Table 2 summarizes the effects of concentration, flow rate and heat treatment on both the magnetic properties and SAR under a field of 150 Oe at 250 kHz. Data for Fe oxide nanoparticles are also given for comparison. It can be seen from Table 2 that the magnetic properties and particle size can be altered continuously by varying the preparation conditions and thermal treatments, thus making it easier to design nanoparticles having a certain set of end-properties. The M_{S} of Fe/Fe₃O₄ particles (130-190 emu/g) was twice as high as Fe oxide alone, and the H_{C} was tunable from several Oe to several hundred Oe.

The difference in magnetization of the Fe/Fe₃O₄ nanoparticles from the Fe bulk value (210 emu/g) may be due to either the presence of nonmagnetic surface oxides dead layers (Chantrell, et al. (1980) J. Phys. D: Appl. Phys. 13:1119) or the canting of moments in the oxide coating. Except for the slow NaBH₄ flow rate sample with a large particle size, all the nanoparticles of 40-50 nm had high H_{C} values from 288-617 Oe, which is nearly an order of magnitude larger than the bulk Fe and Fe oxides values (Chen (1977) Magnetism and Metallurgy of Soft Magnetic Materials, North-Holland, p. 132). The H_{C} of the fine particles can not be explained by assuming the average values of magnetization and magnetocrystalline anisotropy for Fe and Fe₃O₄. The origin of such a large H_{C} could be partly due to the shell-type particle morphology where the oxide coating is believed to interact strongly with the Fe core and partly due to the large surface effects which are expected in small particles (Gangopadhyay, et al. (1992) Phys. Rev. B 45:9778).

Table 2 also reveals that, regardless of higher M_{S}, only 600° C.-annealed particles and particles produced at a slow NaBH₄ flow rate had low H_{C} (74 Oe and 66 Oe, respectively) and higher SARs than pure Fe oxide. Heating from ferromagnetic particles is essentially due to hysteresis losses and Brownian relaxation losses. For immobilized dry particles, the influence of Brownian losses is negligible. Therefore, the particles that undergo significant magnetization reversal will have high hysteresis losses, and also high SAR. In general, high H_{C} particles, although producing wide B-H loops and, consequently, high heating capability, do so only at high values of the external field (at least the coercive field value), whereas very low H_{C} particles, although responsive to low field strengths, produces low heating. For hysteresis heating therapy under the physiological restrictions, an estimated H_{C} value of ˜80 Oe is desirable.

Although a very slow NaBH₄ flow rate (0.75 ml/minute) produced a suitable H_{C} value for a high SAR, this particle was relatively large (more than 200 nm). On the other hand, annealing at 600° C. to decrease H_{C} and retain the nanoscale of the particles achieved a suitable H_{C}. Nevertheless, since the single domain size of Fe is ˜20 nm (Gangopadhyay, et al. (1992) supra), and all the samples had particle sizes ≧40 nm, the particles most probably had a magnetic multi-domain structure. Thus, the magnetization reversal can not be described by Stoner-Wohlfarth model (Stoner and Wohlfarth (1948) Philos. Trans. R. Soc. London. Ser., A 240:599) for single domain particles overcoming of a single energy barrier. Switching instead occurred by a nucleation/propagation process, and so less energy was absorbed. Therefore, smaller single domain particles are needed for better heating effects.

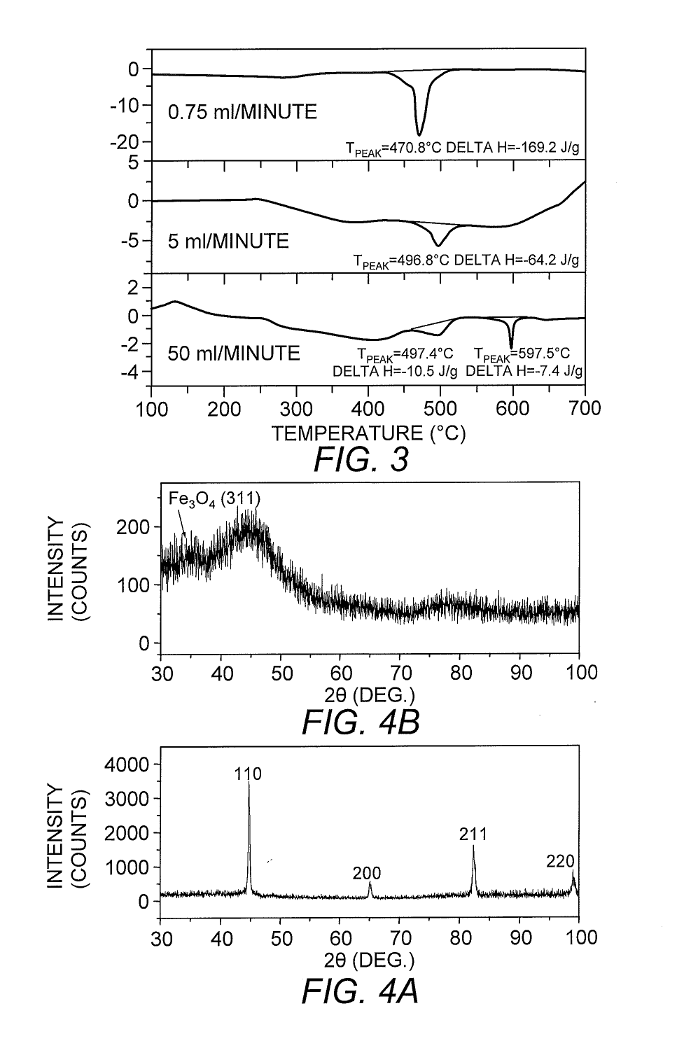

Micro-emulsions were also used to synthesize very small, single domain particles. FIG. 4 shows X-ray diffraction patterns for particles and annealed powders. The particles showed a large band centered at 2θ=44°, the fundamental {110} peak of bcc α-Fe, as well as a possible peak at 2θ=35°, the fundamental {311} peak of f.c.c. Fe oxide. After the powder was annealed under Ar at 500° C. for 5 minutes, the X-ray diffraction spectrum showed only the characteristic pattern of bcc Fe metal, no other peaks or impurities were detected. The disappearance of Fe oxide peak may be because of its very low intensity compared with those of the Fe peaks.

Bright-field TEM micrograph analysis indicated that the particles had a narrow size distribution ranging from ˜10-15 nm. Since the particles are smaller than the critical domain size for Fe (Gangopadhyay, et al. (1992) supra), all the particles should be magnetically single domain. One of the more noteworthy features on the micrographs was the presence of the shell structure revealed as concentric rings on the particles and that many of the particles appeared not to touch their neighbors. This could be attributed to either a thin surfactant coating, or due to Fe/Fe oxide core shell type of structure. Nevertheless, EAD pattern from only several individual particles gave a composite diffraction pattern, bcc α-Fe+f.c.c. Fe₃O₄.

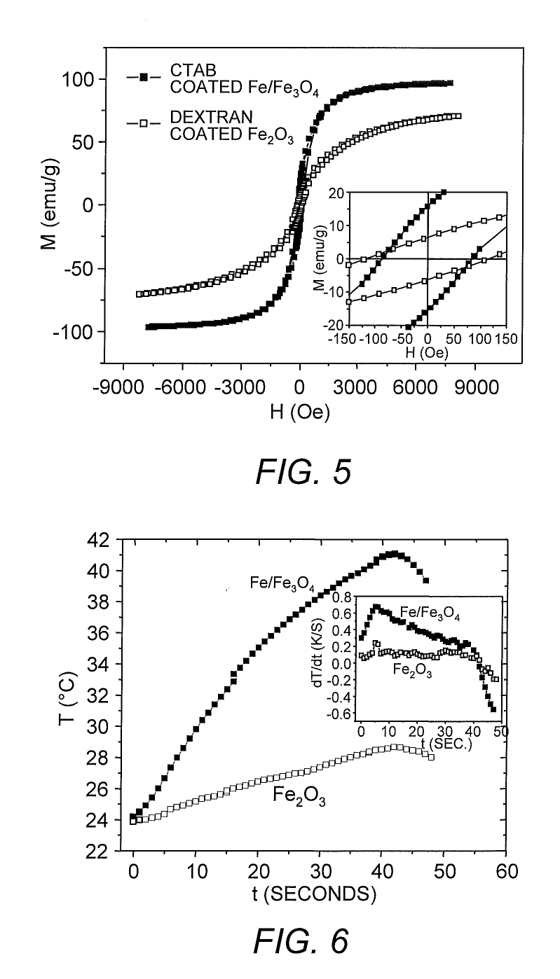

FIG. 5 shows the hysteresis loops for CTAB-coated Fe/Fe₃O₄ powders and Dextran-coated Fe₂O₃ powders at room temperature. The 10-15 nm CTAB-coated dry powder showed obvious hysteresis, i.e., ferromagnetic behavior, as opposed to just superparamagnetic behavior. This again indicated that the Fe/Fe₃O₄ composite had a large effective magnetic anisotropy, i.e., the energy barrier, KV (K the anisotropic constant, V the particle volume), can override the thermal energy, kT (k the Boltzmann constant, T the absolute temperature). Compared to 40-50 nm uncoated Fe/Fe₃O₄ particles, a relatively small of 89 Oe for the 10-15 nm coated Fe/Fe₃O₄ particles was obtained. The origin of this soft magnetic behavior can be explained based on Herzer's random anisotropy model (Herzer (1990) IEEE Trans. Magn. 26:1397) when the particle size is less than the magnetic exchange length. Compared with Fe₂O₃ particles, one can see from FIG. 5 that Fe/Fe₃O₄ particles have higher M_{S}, Mᵣ (magnetic remanence) as well as higher susceptibility. The higher susceptibility could be due to the narrower size distribution, since particles with different sizes have different magnetic anisotropy values.

FIG. 6 shows plots of the temperature rise as a function of time for the CTAB-coated Fe/Fe₃O₄ nanoparticles dispersed in methanol with a concentration of 5 mg/ml under an alternating magnetic field of 150 Oe and 250 kHz. A plot for Dextran-coated Fe oxide particles with the same concentration but dispersed in water is also presented. The temperature rise for the CTAB-coated Fe/Fe₃O₄ particles was much larger than that of Fe oxide particles with the Dextran coating. Correspondingly, the calculated SARs for Fe/Fe₃O₄ particles and Fe oxide alone were 345 and 188 W/g, respectively. The heat capacity of methanol and water were taken as 2.55 and 4.18 J/(Kg), respectively. Heating of ferromagnetic particles was essentially due to hysteresis losses and Brownian relaxation losses. If the influence of Brownian losses is negligible, the particles that undergo significant magnetization reversal will have high hysteresis losses, and also high SAR. In general, high coercivity (HC) particles, although producing wide B-H loops and, consequently, high heating capability, do so only at high values of the external field (at least the coercive field value), whereas very low HC particles, although responsive to low field strengths, produce low heating. A much lower SAR value of 87 W/g for Fe/Fe₃O₄ particles that did not have CTAB coating was noted, which was probably mainly due to their high coercivity. The HC of CTAB-coated Fe/Fe₃O₄ nanoparticles and the Dextran-coated Fe oxide were similar. The difference in SAR between these two types of particles could arise from two factors: the higher M_{S} and the narrow particle size distribution of the Fe/Fe₃O₄ particles. The former directly leads to high hysteresis loop area, the latter leads to a more square loop and therefore larger loop area. It is worth noting that Dextran-coated Fe oxide particles with a similar size distribution (10-15 nm) showed superparamagnetic behavior, but exhibited an even smaller SAR value.

In order to compare the data measured by different authors using different alternating-field parameters, normalization of results have been suggested (Andrä (1998) in Magnetism in Medicine, Andrä & Nowak (ed.), Wiley, Berlin, p. 455) by using a parameter Q, defined as: Q=Hf/(Hf)_{cr}, where H is the applied magnetic field strength at a frequency f. The numerator has the experimentally used values, while the product (Hf)_{cr} in the denominator is the limiting value of Hf above which excessive eddy current heating of tissue occurs. This value of (Hf)_{cr} was experimentally determined by to be 4.85×10⁸ A/ms (Brezovich (1988) Med. Phys. Monograph 16:82). For the present CTAB-coated nanocomposite, SAR/Q=56 is among the highest values reported in the literature. However, a very high value of SAR on the order of 600 W/g at 400 kHz and 137 Oe (SAR/Q=64) has been reported for Fe oxide nanoparticles that were subjected to a magnetic separation technique to produce extremely narrow size distribution (Hergt, et al. (2004) J. Magn. Magn. Mater. 270:345). It is posited that increasing the M_{S} by increasing the particle size within the single-domain range (˜20 nm; Gangopadhyay, et al. (1992) Phys. Rev. B 45:9778) and narrowing the size distribution would increase SAR of the Fe/Fe₃O₄ nanoparticles further.

### Example 3

### Magnetic Resonance Imaging

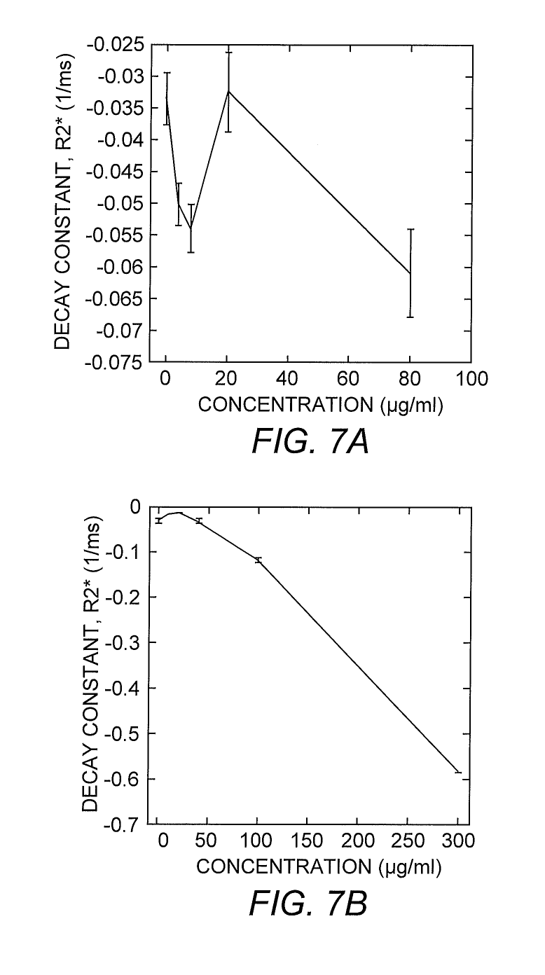

It is important to be able to image the nanoparticle distribution to identify the locations that should be treated and differentiate them from the locations where nanoparticles collect normally, such as the liver. The most commonly employed contrast mechanism is the fact that nanoparticles increase the transverse relaxation rate of the adjacent water in gradient echo images, which creates darker regions in the image at their location. To test the imaging characteristics of the instant nanoparticles, vials of the CTAB-coated Fe/Fe₃O₄ particles and Dextran-coated Fe oxide and particles with different concentrations were imaged in a 3 T Philips Achieva MRI using a pair of 4 inch local pickup coils to achieve the highest signal-to-noise possible. Three-dimensional gradient echo images were obtained with constant TR and variable TE values to calculate the R2* decay constant for each concentration and type of nanoparticles. The 256 by 102 pixel images had isotropic 1 mm voxels. Four values of TE were used: 3.5, 8.0, 12, and 16.1 ms. The TR was 100 ms and the flip angle was 30°. FIG. 7 shows that the R2* decay constant generally increased with increasing concentration, and that the iron oxide nanoparticles (FIG. 7A) had decay constants that were significantly smaller than the new Fe/Fe₃O₄ composite nanoparticles (FIG. 7B). The slope of the linear fit of R2* to nanoparticle concentration was used as the best metric charactering the ability of the nanoparticles to generate contrast in vivo. The variance weighted linear least squares fits produced slopes that were 3.7 times larger for the composite nanoparticles (p value of 3×10⁻⁵): −0.00092 for the composite nanoparticles and −0.00025 for the iron oxide nanoparticles. These data indicate that the Fe/Fe₃O₄ nanoparticles have a significantly higher MRI contrast capacity as compared to the iron oxide nanoparticles.

### Example 4

### Surface Engineering of Iron/Iron Oxide Nanoparticles

Experimental. Fe/Fe₃O₄ core-shell nanoparticles were synthesized through the reduction of aqueous FeCl₃ by aqueous NaBH₄ via combination of two water-in-oil microemulsion solutions (Zeng, et al. (2007) Appl. Phys. Lett. 233112; Zeng, et al. (2007) Proceedings of the SPIE 6440, paper no. 64400H). Each microemulsion solution contained a surfactant: cetyl trimethyl ammonium bromide (CTAB); a co-surfactant: n-butanol; an oil phase: n-octane, and a water phase. Water phases were 0.20 M FeCl₃ and 0.80 M NaBH₄, respectively. To prepare the microemulsions, CTAB was combined with n-octane and n-butanol in two flasks. FeCl₃ and NaBH₄ water solutions were prepared separately and then mixed with oil phase in the flasks. In each microemulsion solution, the molar ratio between the surfactants and oil phase was fixed as: CTAB:n-butanol:n-Octane=1:4.1:11.7, and the volume ratio between oil phase and water phase (O/W) was varied from 7:1 to 1.3:1, for the synthesis of iron nanoparticles with different sizes and magnetic properties.

The microemulsion solutions were agitated for 45 minutes under Ar. Subsequently, the NaBH₄ microemulsion solution was added to the same volume of FeCl₃ microemulsion solution slowly over ˜10 minutes under high speed agitation and then left to react for a further 10 minutes. The resulting nanoparticles were separated using either a centrifuge or a magnetic field, followed by washing with de-gassed de-ionized (DI) water (three times) and methanol (twice).

The next step was the passivation procedure to generate the core-shell structure of iron nanoparticles. The nanoparticles were dispersed in 0.5 wt % trimethylamine N-oxide ((CH₃)₃NO) isopropyl alcohol solution, and sonicated for 30 minutes. After sonication, the nanoparticles were rinsed with methanol and dried under flowing Ar. Finally, the nanoparticles were placed in a desiccator that was filled with Ar for two days to enhance the protective oxide layer for passivation. (CH₃)₃NO works as a mild oxidant that can thicken the crystalline Fe₃O₄ shell on the iron core (Peng, et al. (2006) J. Am. Chem. Soc. 128:10676) or can totally oxidize the iron nanocrystallites to iron oxide nanoparticles (Hyeon, et al. (2001) J. Am. Chem. Soc. 123:12798). Two days passivation of iron particles in Ar is also a critical step to improve the stability of iron nanoparticles. This two-step oxidization procedure enables the iron nanoparticles to be produced and oxidized reproducibly, resulting in reproducible magnetic properties.

The subsequent surface engineering of the nanoparticles included three procedures: CTAB coating removal; surface silanization with hydrophobic hexamethyldisilazane (HMDS); and finally modification with a biocompatible phospholipid coating. For removing the CTAB surfactant on the surface, 100 mg of nanoparticles were sonicated in 10 ml isopropanol and tetramethyl ammonium hydroxide solution (volume ratio 3:1) for 25 minutes at room temperature. After washing using isopropanol and then methanol, the sample was dried under flowing Ar. Next, the iron nanoparticles were silanized using the HMDS. 100 mg of nanoparticles were dispersed in 10 mL 1.0 vol % HMDS toluene solution in a glass vial which was filled with Ar. The sample was then discontinuously sonicated at 50° C. for 4 hours. The nanoparticles were then separated and carefully dried at 90° C. for 2 minutes under Ar.

To obtain a biocompatible coating on the nanoparticles surface, phosphatidylcholine (PC) was assembled onto the nanoparticles surface after the silanization. First the PC was diluted with chloroform to form a 50 mg/ml solution. Silane-modified nanoparticles were dispersed in the desired volume of PC solution and agitated at room temperature for 30 minutes, assisted by discontinuous sonication. Finally, the nanoparticle suspension was dried using flowing Ar to evaporate the chloroform solvent. The PC-coated nanoparticles were stored at 4° C. in a refrigerator.

A FEI Tecnai F20 field emission gun transmission electron microscope (TEM) operated at 200 KV was used to examine the shape and size of the nanoparticles. X-ray diffraction (XRD) measurements were performed using a Rigaku D/MAX diffractometer with Cu-Kα radiation.

The quasi-static magnetic properties were characterized (saturation magnetization, Ms, and coercivity, Hc) from hysteresis loop measurements using a Lakeshore model 7300 vibrating sample magnetometer (VSM). The surface coating was characterized using infrared spectra obtained using a Nicolet Avatar FTIR 330 apparatus with an attenuated total reflection (ATR) unit.

Heating tests were performed using a Hafler power amplifier to drive a resonant network composed of a copper coil and capacitors used to achieve a real input impedance matched to the amplifier for maximum efficiency. A TEKTRONIX 60 MHz AC probe was used with an Agilent Infinium digital oscilloscope to measure the current. Details of the heating set-up and measurements are known in the art (Baker, et al. (2006) J. Appl. Phys. 99:08 H106; Zeng, et al. (2007) supra).

Results. The microemulsion method was an important approach to obtain nanoparticles with a narrow size range and uniform chemical and physical properties. Many nanosized materials can be synthesized by either chemical reduction of metal ions or via co-precipitation reactions in microemulsions (Pileni (1997) Langmuir 13:3266). Iron nanoparticles were produced herein by reducing ferric chloride with sodium borohydride in microemulsions. The reaction can be represented by Eq. (1):

2FeCl₃+6NaBH₄+18H₂O→2Fe↓+21H₂↑+6B(OH)₃+6NaCl (1)

TEM images of iron nanoparticles obtained from the microemulsion with a O/W ratio 7:1 showed that the nanoparticles had an average diameter of ˜8 nm, while nanoparticles obtained from the microemulsion with a O/W ratio 2.5:1 had an average diameter of ˜16 nm. Under high magnification, it was observed that the core-shell structure of the nanoparticles, after passivation in an inert atmosphere, had a 2-3 nm thick iron oxide (Fe₃O₄) layer formed on the Fe particle surface. Electron diffraction patterns of the nanoparticles indicated that the nanoparticles had rings corresponding to both bcc α-Fe and the inverse spinel-structured Fe₃O₄, wherein (110), (200), (211) reflections of α-Fe and (220), (311), (511) reflections of Fe₃O₄ could be clearly distinguished.

Oxidation of iron is strongly dependent on experimental parameters such as temperature, passivation time, oxygen partial pressure, iron particle size and surface situation of iron. These parameters influence the thickness and composition of the oxide layer. Some differences have been reported concerning the crystal structure of the oxide shell. Typically, the oxide layer has been reported to be either magnetite (Fe₃O₄) (Peng, et al. (2006) supra) or a mixture of magnetite and maghemite (γ-Fe₂O₃) (Signorini, et al. (2003) Phys. Rev. B 68:195423), wherein thick iron oxide layers may contain multiple oxide layers, i.e. Fe: FeO: Fe₃O₄: Fe₂O₃ (Wang, et al. (2005) J. Appl. Phys. 98:094308). Fe₃O₄ and γ-Fe₂O₃ have a similar spinel crystal structure with only a small difference in the lattice constants. As described herein, the newly-produced iron oxide shell on iron core nanoparticles was Fe₃O₄. However, with extended exposure to water or after long storage times in air, the lattice parameter was closer to that of γ-Fe₂O₃. This indicated that the iron oxide shell may have a tendency to change from magnetite to maghemite given sufficient time. In so far as the iron nanoparticles were analyzed soon after the passivation, the composition of the iron shell in this example was found to be Fe₃O₄.

The average particle size of the iron nanoparticles was measured using TEM and expressed as a function of the O/W ratio in the microemulsions. This analysis indicated that particle size decreased with increasing O/W ratio from 20 to 8 nm as the O/W ratio increased from ˜1.3 to 7.0. When the O/W ratio was high, the molar ratio of surfactant to water was also increased, resulting in a high surface tension at the oil/ater interface. This produced small water droplets and defined the iron nanoparticle size.

Magnetic properties of the nanoparticles were also determined an expressed as a function of nanoparticle diameter. Both the saturation magnetization and coercivity increased with increasing iron particle size: M_{S} increased from 36 to 113 emu/g and the Hc increased from 14 to 185 Oe as the particle diameter increased from 8 to 20 nm. The iron composite nanoparticles were all less than 20 nm and, thus, their inner iron cores were considered to be single domain. The saturation magnetization arose from both the iron core (218 emu/g), and the iron oxide shell (for Fe₃O₄, 80-92 emu/g), based on the relative weight percentage of iron, iron oxide and the non-magnetic coatings on the particle surface. For particles having a similar shell thickness, the weight ratio of the iron core to the iron oxide shell was greater for large particles than for small particles. This was the reason for the higher M_{S} of the larger particles. It was contemplated that the very thin iron oxide shell may not have contributed to M_{S} due to its superparamagnetic behavior. Meanwhile, the specific surface areas were inversely proportional to the particle radius. Thus, the low M_{S} value of the small iron particles was also from the higher absorption of non-magnetic coating on their surface. The increasing coercivity with increasing particle diameter (for small particles) was expected based on the random anisotropy model (Herzer (1990) IEEE Trans. Magn. 26:1397). However, when the size of Fe particles was less than ˜8 nm, they probably become superparamagnetic producing no magnetic moment at room temperature.

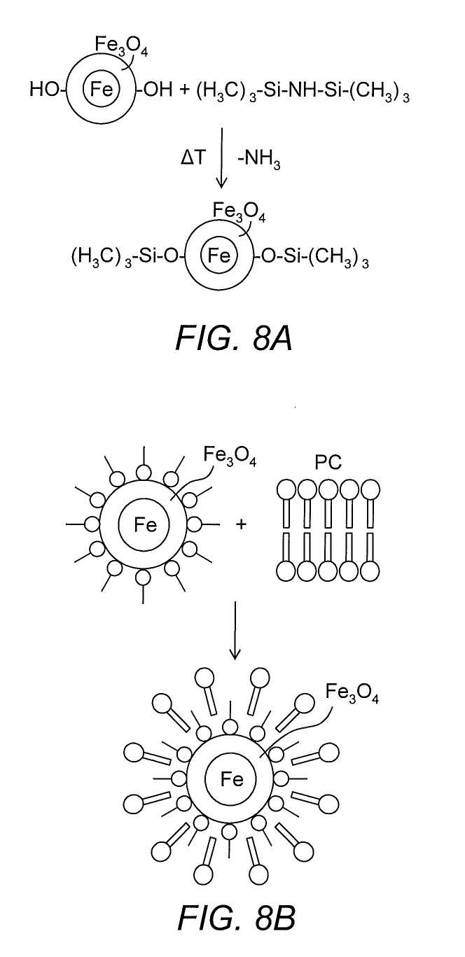

CTAB is an ionic surfactant that has strong positive change and can be strongly absorbed onto the iron nanoparticle surface via its headgroup (Dobson, et al. (2000) Vibr. Spectrosc. 24:287) during the synthesis process. CTAB serves as a good protection layer for the iron nanoparticles preventing the oxidation of iron during its passivation and storage. However, it was contemplated that the CATB coating may contain some residuals from the microemulsion and by-products from the reaction, such as B(OH)₃, octane and butanol, thereby limiting the range of applications of the nanoparticles. Usually, a surfactant can be removed by strong washing, sintering (Brimaud, et al. (2007) J. Electroanal. Chem. 602:226) or replacement by other reaction agents or surfactants (Jeunieau & Nagy (1998) Appl. Organometal. Chem. 12:341; Xie, et al. (2007) Adv. Mater. 19:3163). For the analysis herein, the CTAB coating on the nanoparticles was removed using a mixture of TMAH and isopropanol. TMAH is a strong organic base. In photolithography, it is used as both the developer and the stripper that removes the photoresist and polymer residuals (Reichmanis, et al. (1989) American Chemical Society, Washington, D.C.). Also, it can improve the dispersion of the nanoparticles in aqueous solution (Salgueirino-Maceira, et al. (2004) Langmuir 20:6946). HMDS silanization is a moderate method to obtain a hydrophobic monolayer on the sample surface and this modification process can be completed either in the vapor or liquid phases. After modified by HMDS silane, a thin hydrophobic layer covers the Fe/Fe₃O₄ nanoparticle surface. This thin layer enhances the linking between the nanoparticle surface and phospholipid, and also improves the stability of the iron nanoparticle in aqueous solution. The principle of HMDS silanization is shown in FIG. 8A. The phosphatidylcholine incorporates choline as a hydrophilic headgroup and the fatty acids as the hydrophobic tail in its structure. HMDS-coated Fe/Fe₃O₄ nanoparticles are further modified with PC through the mechanism shown in FIG. 8B. The hydrophobic Van Der Waals interaction between the hydrophobic tail of PC and the hydrophobic surface of nanoparticle forms a thermodynamically-defined interdigitated bilayer structures surrounding each nanoparticle (Peng, et al. (2006) Pure Appl. Chem. 78:1003). PC coating provides a biocompatible surface for the iron nanoparticles and allows them to be well dispersed in aqueous solutions.

The surface modification results in changes in the magnetic properties of the iron nanoparticles due to the oxidation of iron that occurs during the modification process, mostly during the CTAB removal step. The oxidation of the iron nanoparticles is reflected in their change in saturation magnetization. The original CTAB-coated iron particles had an M_{S} of 104 emu/g and H_{C} of 180 Oe. The same sample after CTAB removal by TMAH had M_{S} and H_{C} values of 73 emu/g and 119 Oe, respectively. In contrast, the sample coated with HMDS after the CTAB removal showed slightly higher M_{S} and H_{C} values of 83 emu/g and 152 Oe, respectively.

PC modification was a very moderate self-assembled process, and it was very helpful to keep the iron core intact inside nanocomposites. It did not affect the coercivity of the iron composite nanoparticles. No change in the coercivity of the Fe/Fe₃O₄ nanoparticle was observed after PC modification. However, the M_{S} value decreased due to the extra mass added from the non-magnetic PC. FTIR spectral analysis of the PC-coated iron composite nanoparticles indicated bands around 817, 966, 1076 and 1243 cm⁻¹, which were attributed to the —PO₄³⁻ group vibration mode. The bands observed at 1390 and 1460 cm⁻¹ were due to bending vibration of —CH₂— group. The two bands at 2853 and 2925 cm⁻¹ were related to vibrations of symmetric and asymmetric methylene group and methyl group.

The stability of iron nanoparticles depended on their particle size, surface condition and environment. The Fe/Fe₃O₄ nanoparticles became chemically unstable in aqueous solution, especially when they were small and had a hydrophilic surface. Fe/Fe₃O₄ nanoparticles (with diameters large than 10 nm) synthesized from microemulsion after careful passivation were stable in air for more than 3 months without obvious changes in their magnetic properties. TEM images of the Fe/Fe₃O₄/HMDS/PC nanoparticle before and after dispersion in water for 1.5 hours showed that the water-exposed nanoparticles still maintained metallic iron cores.

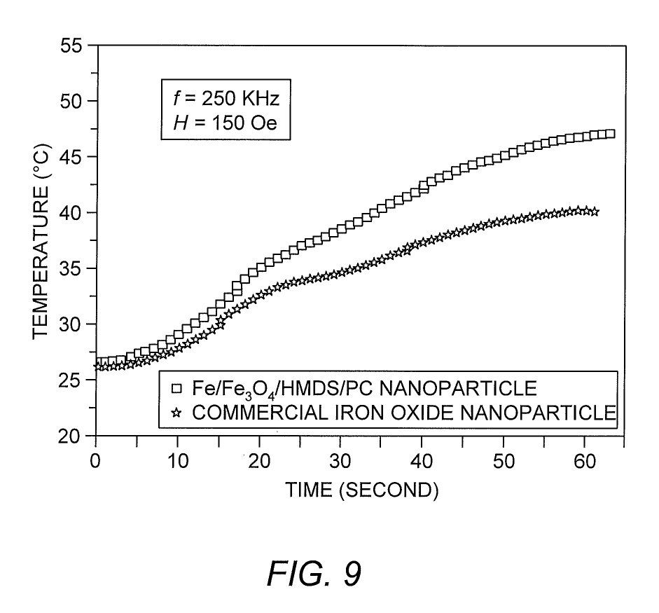

FIG. 9 shows a plot of the temperature rise as a function of time for the instant Fe-based nanoparticles dispersed in DI water with a concentration of 6 mg/ml under an alternating magnetic field of 150 Oe at a frequency 250 kHz. The M_{S} and H_{C} values of Fe/Fe₃O₄/HMDS/PC nanoparticles for the measurement were 55 emu/g and 69 Oe, respectively. Moreover, in the nanoparticles, the weight ratio between the PC coating and the Fe/Fe₃O₄/HMDS core was 0.3:1.0. Fe-based nanoparticles showed much greater heating compared to Dextran-coated Fe oxide particles. The heating effect strongly depends on the magnetic particle's properties, measurement conditions, particle size distribution and particle dispersion (Hergt, et al. (2006) J. Phys.: Condens. Matter 18:S2919). Meanwhile, the stability of the particles also plays an important role. The iron core in the composite nanoparticle of this invention provided high magnetic saturation and a large hysteresis loop resulting in a substantial amount of heat produced by the nanoparticles.

Accordingly, the composite nanoparticles of this invention present higher stability in aqueous solution while still maintaining good magnetic properties. With the thin hydrophobic silane layer protection, the oxidation of iron was greatly reduced, giving iron nanoparticle enough life time in aqueous environment for biomedical applications. Compared to iron oxide nanoparticles, the iron core in this composite nanoparticle provides high magnetization and increased hysteresis losses, enhancing its use for hyperthermia.

What is claimed is:

1. A nanoparticle composition comprising an iron core and an iron oxide shell.

2. The nanoparticle composition of claim 1, further comprising a surfactant.

3. The nanoparticle composition of claim 1, further comprising a silane layer on the surface of the nanoparticle.

4. The nanoparticle composition of claim 3, further comprising a biocompatible phospholipid coating on the surface of the silane layer.

5. A method for producing a nanoparticle composition, comprising
reducing aqueous FeCl₃ within a NaBH₄ solution so that an iron core is formed and
passivating the iron core to produce an iron oxide shell so that a nanoparticle composition with a metallic iron core and an iron oxide shell is produced.

6. The method of claim 5, wherein the step of reducing aqueous FeCl₃ within a NaBH₄ solution further comprises a surfactant.

7. The method of claim 6, further comprising removing the surfactant from the nanoparticle and silanizing the nanoparticle.

8. The method of claim 7, further comprising coating the nanoparticle with a biocompatible phospholipid.

9. A nanoparticle produced by the method of claim 5.

10. A method for hyperthermia treatment of cancer comprising administering an effective amount of the composition of claim 1 to a subject with cancer and exposing the subject to a magnetic field thereby effecting hyperthermia treatment of the cancer in the subject.

11. A method for hyperthermia treatment of cancer comprising administering an effective amount of the nanoparticle of claim 9 to a subject with cancer and exposing the subject to a magnetic field thereby effecting hyperthermia treatment of the cancer in the subject.

12. A method for imaging cancer comprising administering the composition of claim 1 to a subject with cancer and detecting the localization of the nanoparticle thereby imaging the cancer in the subject.

13. A method for imaging cancer comprising administering the composition of claim 9 to a subject with cancer and detecting the localization of the nanoparticle thereby imaging the cancer in the subject.
